(19) United States
(12) Patent Application Publication
(10) Pub. No.: US 2005/0038420 A1
(43) Pub. Date: Feb. 17, 2005

(54) Cooling cannula system and method for use in cardiac surgery

(76) Inventors: M.A.J.M. Huybregts, Amstel (NL); Robert J. Todd, Salt Lake City, UT (US); Alison S. Curtis, Riverton, UT (US)

Correspondence Address: EDWARDS LIFESCIENCES LLC;Law Department, One Edwards Way, Irvine, CA, 92614, US

(21) Appl. No.: 10/441,772

(22) Filed: May 19, 2003

Related U.S. Application Data
(63) Continuation-in-part of application No. 10/151,354, filed on May 20, 2002, now abandoned.

(51) Int. Cl. A61M 37/00; A61B 18/18

(52) U.S. Cl. 606/20; 604/6.13

(57) ABSTRACT

A novel improved enhanced cardiac surgical method yields unexpected results by having an enhanced intraluminally emplaced cooling system. In preferred device embodiments improvements include a first means for draining venous blood from at least one of the right atrium, superior vena cava and inferior vena cava and an improved means for cooling involved luminal surfaces. Tissue insult and injury is substantially mitigated by engagement of the cooling means with select aspects of involved atrial tissue to augment transfer of heat. In one embodiment of the invention, the right atrium is cooled while the patient's body is maintained at a normothermic temperature during surgery. The alternate cooling mechanisms disclosed have applicability both on- and off-pump in a variety of procedures ranging from traditional open cardiac surgical repair and by-pass to endovascular procedures using percutaneous access and minimally invasive therapies.

### RELATED APPLICATIONS

This application is a continuation-in-part of U.S. patent application Ser. No. 10/151,354, entitled “Cooling Cannula System and Method for Use in Cardiac Surgery,” filed May 20, 2002, which is incorporated herein in its entirety.

### FIELD OF THE INVENTION

This invention pertains generally to medical devices and their methods of use for treatment of cardiovascular disease, and more particularly to specialized cannulae useful for implantation and/or emplacement within cavities or blood vessels of the body. The cannula of the present invention is particularly suited to mitigate, extenuate or otherwise positively impact cellular or tissue based insult during any cardio thoracic, cardiovascular, or related procedure by facilitating heat transfer away from the body tissue.

### BACKGROUND OF THE INVENTION

Current technology addresses needs within the context of the world's most prevalent set of disease states, namely those loosely grouped under the term cardiovascular disease. It is respectfully proposed, however, that aspects of the instant teachings bridge the gaps between current therapies targeting cardiac surgery in such a way that artisans will readily ascertain and understand the cross-functional utility of the instant teachings. Likewise, it is further submitted that novel approaches set forth herein are expressly de-limited to provide improvements with any appropriate groups of procedures within known or developed therapies used as stand alone or adjunct to conventional cardiac, thoracic and vascular surgery.

The human heart is normally slightly larger than a clenched fist. It is cone shaped in appearance, with the broad base directed upward and to the right and the apex pointing downward and to the left. The human heart is located in the chest (thoracic) cavity. It is situated behind the breastbone (sternum); in front of the windpipe (trachea), esophagus, and descending aorta; between the lungs; and above the diaphragm. The heart is divided by partitions (septa) into right and left halves, and each half is divided by septa into two chambers. The upper chambers are the atria, and the lower chambers are the ventricles. The right atrium is a thin-walled chamber receiving blood from all tissues except the lungs. Three veins empty into the right atrium: the first two, which bring blood from the upper and lower parts of the body, are the superior vena cava (SVC) and the inferior vena cava (IVC). The third, the coronary sinus, drains blood from the heart itself. Blood flows from the right atrium to the right ventricle, which in turn pumps blood into the pulmonary artery and ultimately to the lungs.

The left atrium receives the four pulmonary veins, which bring oxygenated blood from the lungs. Blood flows from the left atrium into the left ventricle. Blood is then pumped from the left ventricle through the aorta to all parts of the body except the lungs.

prevent backflow of blood, the heart is equipped with valves that permit the blood to flow in only one direction. The atrioventricular valves (tricuspid and mitral) are thin, leaf-like structures located between the atria and ventricles. The right atrioventricular opening is guarded by the tricuspid valve, whereas the mitral valve guards the left atrioventricular (AV) opening. The semilunar valves are pocket-like structures attached at the point at which the pulmonary artery and the aorta leave the right and left ventricles, respectively. While known treatments are effective at addressing surgical needs such as bypass grafting and repair and replacement of defective failed or failing valves, vast room for improvement exists within the macro-context of the surgical procedures themselves, in terms of the impact on crucial cellular, tissue-based and organ system level insults.

The heart possesses a vascular system of its own, called the coronary arterial system. It comprises two major coronary arteries the right and left coronary arteries. These arteries originate from the right and left aortic sinuses (the sinuses of Valsalva), which are bulges at the origin of the ascending aorta immediately beyond the aortic valve. Venous blood from the heart is carried through veins to the coronary sinus, which empties into the right atrium between the IVC and the AV orifice.

The pacemaker of the heart is the sinoatrial node (SA node). This highly important structure is a small strip of specialized muscle located in the posterior (back) wall of the right atrium, immediately beneath the point of entry of the SVC. After an action impulse is generated by the SA node, the impulse immediately spreads through the atrium and is relayed to the atrioventricular node (AV node), located in the lower part of the right rear atrial wall. The coordinated functioning of the SA node and the AV node are responsible for the regulated contractions of the normal heart.

A leading source of mortality in Western-style societies is the above referenced general category of cardiovascular disease. Significantly, the categories of each of heart ventricular dilation (HVD), congestive heart failure (CHF), and coronary artery disease (CAD remain) prominent and ostensively seem to be manifested in ever growing segments of the patient populace. It is becoming apparent that there is often a strong correlation between these three categories and peripheral vascular disease (PVD), and those showing signs of at least one likely have symptoms of the other three.

CAD can be manifested in a number of ways. The risks and discomfort associated with angina and ischemia can be produced by the impaired blood flow resulting from CAD. In HVD, CHF, and CAD clinicians are seeing instances of major adverse cardiac events such as, but not limited to, myocardial infarction resulting from acute blockage of coronary blood flow, producing damage to myocardial tissue, death, stroke and other lifestyle destructing results and eventualities.

Treatment of HVD, CHF, and CAD has been accomplished through a plurality of different approaches. Pharmacological treatment of early symptoms with medicines or diet and lifestyle modification is a first step designed to address the underlying disease process. Often this must complement both surgical techniques and endovascular treatment of the coronary blockage, which may be accomplished with balloon angioplasty, atherectomy, laser ablation, stenting, and similar devices.

When pharmacological intervention or endovascular treatment do not fully address the issues, coronary artery bypass grafting (CABG) procedures may become necessary. Worldwide, more than 500,000 patients suffering from heart disease are annually afforded the benefits of therapeutic CABG surgery.

CABG uses lengths of superficial veins from the legs which are inserted between the aorta and a part of a coronary artery below the obstruction. Multiple grafts are often used for multiple occlusions. Such multiple grafts are referred to as “triple bypass” or “quadruple bypass” operations, for example. The internal mammary arteries can also used to provide a new blood supply beyond the point of arterial obstruction; however, since there are only two internal mammary arteries, their use is limited.

In undertaking CABG surgery, cardiopulmonary bypass (CPB) is often used. Here, the goals are to provide life support functions for the patient, and to provide a motionless, decompressed heart, as well as a dry, bloodless surgical field for the surgeon. CPB may be accomplished by use of large drainage tubes (cannulae and catheters) inserted in the right atrium, superior and/or inferior venae cavae. CPB may be done by the establishment of a heart-lung life-support system that provides a diversion of oxygen-poor blood from the venous circulation and its transport to a heart-lung machine. There, re-oxygenation and carbon dioxide elimination are accomplished. Additionally, heat transfer-either warming or cooling-of the diverted bloodstream is provided. The oxygenated blood is then returned to the arterial system via aortic cannulae.

“On-pump” procedures work whereby processed blood streams are then selectively pumped back to the body and returned to the arterial system through cannulae introduced in a major systemic artery, such as the aortic or femoral artery. Meanwhile, the heart may be opened and the corrective operation performed. This procedure permits a surgeon to operate on a relatively motionless heart for many hours, if necessary.

It is further noted that on the continuum of surgical procedures and involved in pump and “off pump” procedures many different approaches have come to be important or had clinical significance, or likely shall. To these ends, the instant teachings are understood to impact both sub-generic types of procedures, and one of ordinary skill in the art will readily understand these uses.

In terms of structure being driven by function, it has become known that the details of the design and construction of cardiac catheters for these procedures is obviously of great importance to their success. Such catheters can, for example, be inserted via the right atrium or via a peripheral vein such as the jugular vein. But the direct insertion of catheters into the right atrium can result in direct surgical trauma from the holes cut into these structures for catheter entry. Such trauma can lead to bleeding, cardiac arrhythmias, air embolism and surgical adhesions.

A solution to some of these problems was provided by M. A. J. M. Huybregts in U.S. Pat. No. 5,562,606 by providing cooling means around a cannula adapted to be inserted into the superior vena cava, through the right atrium and into the inferior venae cavae. Cooling is generally a protective measure which lowers the metabolic needs of the organ or tissue being cooled. As a result, the insult to the area is mitigated. Namely, it is thought that specific cooling of the right atrium will prevent the insult to those tissues and result in reduced ischemic injury, reduced tissue death, reduced inflammatory response, and ultimately reduced disturbances like atrial fibrillation.

The Huybregts cooling means, however, were limited to aspects of an inflatable balloon adapted to lie against the inside wall of the right atrium and along the transitions of the vena cava in the right atrium. Alternate cooling means, ranging from chemical to electrical to biochemical to evaporative may exist providing an unexpected benefit to these disclosures. The Huybregts' 606 patent is incorporated by reference herein. It is proposed that the instant teachings bring the Huybregts solution to another level, with unexpected results.

Likewise clinically in surgical practice, several limitations were found in the use of such balloons as the only cooling method. First, the balloon was found to interfere with the manipulation of the heart required for surgery on the backside coronaries. In order to access the posterior portion of the heart for surgical procedures, it is necessary to “flip”, or rotate the heart so that the posterior portion is exposed. In practice, the axis for this rotation is the cannula itself.

Using cannulae of the prior art, it has been found that there is a possible risk of damaging structures within the heart during this “flipping” maneuver. This procedure creates difficulty with access to posterior grafts. Prior art devices were also found to be deficient in producing a leak-free isolation of the right atrium. Further, prior art devices were found to be deficient in contacting and therefore cooling the sino-atrial node. Still further, prior art devices have been found to produce a possible risk of blockage of the coronary sinus. Even further, prior art devices have been found to produce a possible risk of infringing and hence damaging the tricuspid valve. Even yet further, cooling balloons of the prior art have been found to have an expanded conformation wherein the middle portion of the balloon has a larger circumference than the end portions. Such an expanded conformation combined with deficient leak-free isolation of the right atrium can result in distention of the atrium during surgery, producing undesirable damage to anatomical structures.

With prior art devices such as dual stage cannula, the isolation of the right atrium is not attempted and venous blood is allowed to backfill the right atrium through the great vessels and the coronary sinus. The result is a right atrium at the same, warm temperature as the venous blood. In addition, backflow at the coronary anastomoses is expected, which compromises surgical visualization. If the right atrium could be isolated, it would be possible to effectively cool the right atrium as well as eliminate backflow at the distal anastomoses.

In order to achieve right atrial isolation via superior vena cava and inferior vena cava vessel occlusion in a coronary artery bypass grafting procedure, the surgeon typically forms two incisions in the right atrium. The surgeon then advances one cannula into the inferior vena cava and a second cannula into the superior vena cava, tying off the vessels with vessel loops around the vessel/cannula combination thus preventing blood flow into the right atrium. It is important to note that such incisions may damage electrical conductivity pathways and may result in reentry phenomenon ultimately manifesting itself in transient conduction abnormalities like arrhythmias and atrial fibrillation. This approach is also time-consuming and laborious. As a result, it is used for repairing or replacing cardia valves and is not usually used for CABG procedures.

Greater cooling of the right atrium than that which is available with prior art devices would reduce the heart's electrical activity and help to reduce postoperative atrial fibrillation. Likewise, cooling of the sinoatrial node to lower temperatures than is possible with currently employed heat transfer media is highly advantageous in further reducing its electrical activity.

It remains the case that prior devices, products, and methods currently available to medical practitioners have not adequately addressed the need for minimizing deficiencies in cardiac cooling and the potential for damage to cardiac anatomical structures. The present invention addresses the clear need for advanced methods and apparatus for solving the long-standing needs in atrial cooling as set forth above. Those skilled in the art are well versed in the cross-functional approaches and advances ranging from the most to least invasive procedures, on-and off-pump methods and the like treatment schemes by which the instant teachings may be actualized and actuated.

### OBJECTS AND SUMMARY OF THE INVENTION

One aspect of the present invention is to provide novel enhanced means for cooling select aspects of a patient's vasculature whereby cell, tissue and organ insult is mitigated or prevented.

Another aspect of the present invention provides novel enhanced means for cooling select aspects of a patient's cardiac anatomy whereby cell, tissue and general cardiac insult is mitigated or prevented.

Yet another aspect of the present invention provides tubular means for cooling select aspects of a patient's cardiac anatomy whereby cell, tissue and general cardiac insult is mitigated, extenuated or prevented.

A further aspect of the invention applies an improved cooling technique to procedures designated to address, ameliorate, extenuate, mitigate, or prevent tissue insult and injury within the context of cardiac surgery therapies.

Another aspect of the present invention provides a cooling cannula system that facilitates “flipping”, or rotating of the heart, so that the posterior side can be exposed during surgery without damaging structures within the heart.

It is another aspect of the present invention, when designed in combination with a cannula, is to provide a cooling cannula system effective to produce a leak-free isolation of the right atrium.

Another aspect of the present invention provides a cooling cannula system that does not produce a possible blockage of the coronary sinus during surgery.

In another aspect of the present invention, a cardiac surgical procedure is provided whereby the heart may be cooled while the patient's body is maintained at a normothermic temperature.

In another aspect of the present invention a cardiac surgical procedure is provided whereby the right atrium is cooled while the patient's body is maintained at a normothermic temperature.

In another aspect of the present invention a cardiac surgical procedure is provided whereby two or more of the right atrium, SA node and AV node are cooled while the patient's body is maintained at a normothermic temperature during the surgical procedure.

In another aspect of the present invention a cooling cannula system is provided that includes a cooling member having an expanded conformation wherein the middle portion has a circumference no greater than the end portions.

In another aspect of the present invention a cooling cannula system lacking a possible risk of infringing and hence damaging the tricuspid valve is provided.

In another aspect of the present invention a cooling system that can provide cardiac cooling to lower temperatures than is possible with currently employed devices is provided.

In another aspect of the present invention a cooling system is provided that embodies a solid-state thermoelectric cooling device utilizing the Peltier effect.

In another aspect of the present invention a cooling system is provided that can cool the sinoatrial node to lower temperatures than is possible with currently employed techniques and tools.

In another aspect of the present invention a cooling system that effectively reduces right atrial and nodal insult and ultimately reduces post-operative atrial fibrillation is provided.

In another aspect of the present invention a cooling system incorporating a cooling member possessing circumferential bands for shape control is provided.

In another aspect of the present invention a cooling system is provided that, when designed in combination with a cannula, can be adapted for embodiments that are capable of direct insertion via one of the following: the superior vena cava, the inferior vena cava, the right atrium or peripheral insertion via a vein (for example, the jugular or femoral vein).

In another aspect of the present invention a cooling system is provided that, when designed in combination with a cannula, can be adapted for embodiments that are capable of direct insertion via one of the following: the superior vena cava, the inferior vena cava or peripheral insertion via a vein (for example, the jugular or femoral vein), without requiring an incision in the right atrium.

In another aspect of the present invention a cooling cannula system is provided that can isolate the right atrium and eliminate backflow at distal anastomoses.

In another aspect of the present invention a method and apparatus for effectively cooling the heart without unduly lowering the systemic core temperature is provided.

Further objects and advantages of the invention will become apparent to those skilled in the art upon reading and understanding the following detailed description and the accompanying drawings. It should be understood that the drawings are not necessarily to scale, that they show only certain embodiments of the invention, that certain details not essential to an understanding of the invention may have been omitted, and that like numbers indicate like structures.

### BRIEF EXPLANATION OF THE DRAWINGS

Those of skill in the treatment of cardiac surgery will understand that these illustrated embodiments enable, but in no way, limit the instant teachings.

FIG. 1 is a schematic cut-away view of a cooling cannula, according to the invention, inserted via an opening in the superior vena cava;

FIG. 2 is an enlarged, cut-away, elevational view of a cooling cannula system of the present invention;

FIG. 3 is an enlarged cross-sectional view through line a-a of FIG. 2;

FIG. 4 is an enlarged cross-sectional view through line b-b of FIG. 2;

FIG. 5 is an elevational view of a cooling cannula system of the present invention;

FIG. 6 is an elevational view of one embodiment of the cooling cannula system according to the present invention; and

FIG. 7 is an elevational view of a cooling cannula system of the present invention.

### DETAILED DESCRIPTION OF THE PREFERRED EMBODIMENTS

The present invention is directed to improved cooling cannula systems and methods for their use in cardiac surgery. The present invention may exist in numerous embodiments, including those that may be inserted peripherally thus avoiding the need for a major chest incision such a thoracotomy or median stemotomy.

By utilizing fluoroscopic or ultrasound imaging, the cooling device when designed in combination with a cannula may be precisely positioned such that upon inflation of inflatable members such as balloons, the flow of blood into the right atrium is fully blocked thereby achieving total heart bypass. The combined cooling device and cannula may use conventional heat transfer media cooled by refrigeration systems known in the art, or it may use thermoelectric cooling devices to accomplish the desired cooling.

By way of background, and in no way limiting the instant teachings, thermoelectric coolers are solid-state heat pumps that operate on the Peltier effect, the theory that there is a heating or cooling effect when electric current passes through two conductors. A voltage applied to the free ends of two dissimilar materials creates a temperature difference. With this temperature difference, Peltier cooling will cause heat to move from one end to the other. A typical thermoelectric cooler will consist of an array of p- and n-type semiconductor elements that act as the two dissimilar conductors. The array of elements is soldered between two ceramic plates, electrically in series and thermally in parallel. As a dc current passes through one or more pairs of elements from n- to p-, there is a decrease in temperature at the junction (“cold side”) resulting in the absorption of heat from the desired structure. The heat is carried through the cooler by electron transport and released on the opposite (“hot”) side as the electrons move from a high to low energy state. The heat pumping capacity of a cooler is proportional to the current and the number of pairs of n- and p-type elements (or couples).

In accordance with one embodiment of the invention, there is provided a cooling cannula comprising an insertion piece for insertion into the right atrium through the superior vena cava. The insertion piece has a plurality of apertures for drainage of the inferior vena cava at its distal end, and is joined at its proximal end to the distal end of a connection piece. The connection piece is fitted at its proximal end with a coupling to a pump inlet of the heart heart-lung machine. The apertures collectively comprise a cross-section sufficient to accommodate the blood being bypassed.

In one embodiment of the present invention, the cannula cools the right atrium to a temperature below 20° C. In another embodiment of the present invention, the cannula cools the right atrium to a temperature between 8° C. and 17° C. In a still further embodiment of the present invention, the cannula cools the right atrium to a temperature between 10° C. and 15° C.

The above described, currently clinically utilized version of the instant teachings likewise feature a cannula which is preferably bent to form an angle between about a right angle and an obtuse angle of about 110° (an angle of inclination from linearity of about 70°). One skilled in the art would realize that other angles may be desired depending on the patient's vasculature, and surgeon's approach. The insertion piece has a side opening(s) positioned so as to drain the superior vena cava. In other embodiments the device can be constructed to enable insertion either through the superior vena cava, right atrium, inferior vena cava, or peripherally.

In embodiments designed to be inserted through the inferior vena cava, the plurality of apertures drains the superior vena cava, and the side opening(s) drain the inferior vena cava. In embodiments designed to be inserted through the femoral vein, the plurality of apertures drains the superior vena cava, and the side openings(s) drain the inferior vena cava. In embodiments designed to be inserted through the jugular vein, the plurality of apertures drains the inferior vena cava, and the side opening(s) drains the superior vena cava.

The internal diameter of the insertion piece and the internal diameter of the connecting piece are proportional to the volume of the transported bloodstream. The invention provides an improved means for cooling the surfaces of the atrium involved in CPB procedures. In one embodiment, the means for cooling comprise a radially expandable cooling membrane which in one expanded predetermined configuration has substantial engagement with the thick tissue shelves of the right atrium, but does not have substantial contact with the atrial appendages, tricuspid valve, coronary sinus, and other interior surfaces of said right atrium. In this way, damage to the thin tissue appendages, tricuspid valve, coronary sinus, and other interior surfaces of the right atrium is avoided. Moreover, the supraventricular excitor and conduction systems are protected, thus providing intraoperative protection of the cooled tissues and ultimately reducing postoperative conduction disturbances.

This invention provides indirect cooling means for the thin atrial appendages and other interior surfaces of said right atrium. The cooling means, in a preferred embodiment, comprises an inflatable membrane that has a separate inlet and outlet duct to allow a continual flow. A thin layer of liquid or blood between the membrane and the luminal aforementioned structures of the right atrium is sufficient to enable sufficient heat transfer to account for the observed cooling. In this way, damage to the thin atrial appendages and other interior surfaces of the right atrium is avoided.

The heat radiation from the cannula in the right atrium is also absorbed by the inflatable membrane. The inflatable membrane, which is made of a biocompatible polymer such as polyurethane, may be made more conductive by the incorporation of particles of a biocompatible metal, or particles of a biocompatible metal alloy comprising at least two elements selected from the group consisting of iron, cobalt, chromium, nickel, titanium, niobium, and molybdenum. Alternatively, the same metals or alloys can be formed into sheets to coat both the interior and exterior surfaces of said inflatable membrane.

The following describes presently preferred embodiments of the invention only, and is not intended to exhaustively describe all possible embodiments in which the invention may be practiced. As discussed, unique aspects of CAD, HVD, CHF and the like disease states drive modifications of the described devices and methods such the artisans would be able to effect such changes, given the guidance of those descriptions with the claims appealed hereto.

Unless defined otherwise, all technical and scientific terms used herein have the same meaning as is commonly understood by one of skill in the art to which this invention belongs. All patents and publications referred to herein are incorporated in their entirety by reference.

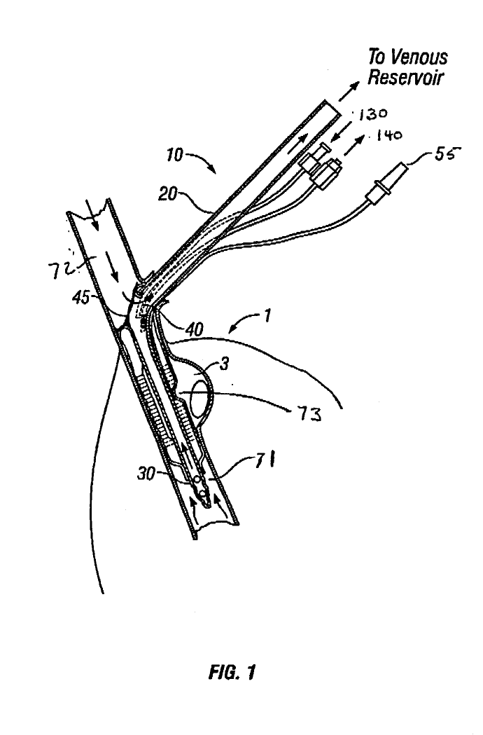

Referring to FIG. 1, a portion of a heart, generally indicated by 1, includes the right atrium 3, the superior vena cava 72, and the inferior vena cava 71. During an operation where heart function is disconnected, and the circulation is taken over by a heart-lung machine, blood is extracted through the right atrium, the superior cavae or the inferior venae cavae.

A cannula 10, typically having an outer diameter between about 10 Fr and about 55 Fr is used for this purpose, and may include fluid communication access adapted for venous blood pressure measurement of said patient. As may be seen, the embodiment of the cannula 10 depicted in FIG. 1 is generally tubular in shape. This tubular shape, which allows the cannula to be the axis of rotation, facilitates “flipping” the heart to provide access to the posterior portion during surgical procedures. The cannula 10 is not designed to fill the right atrium.

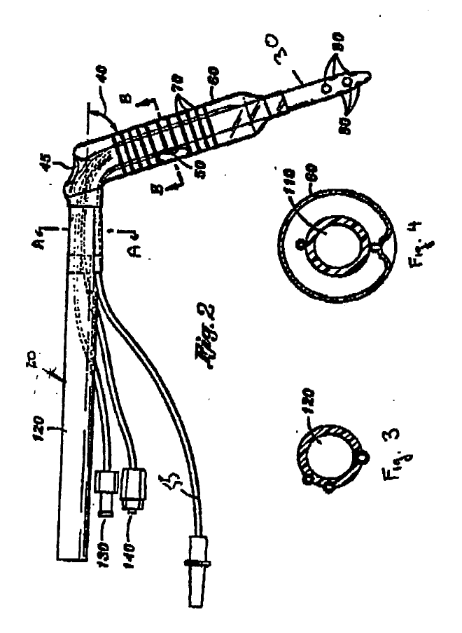

FIG. 2 provides and enlarged, cut-away, elevational view of the cooling cannula system of the present invention.

Cannula 10 consists of a connecting piece 20 and a thinner insertion piece 30. Connecting piece 20 and insertion piece 30 are joined to each other by an angle of inclination 40. Angle 40 can range between approximately 45° and approximately 180° depending on the physical properties of the material used to make cannula 10. When such material is flexible and can be bent without occluding the lumen of the cannula, an angle larger than about 45° is permissible, Preferably, the angle is between about 100° and 120°, and even more preferably between about 105° and about 115°. An opening 45, which provides a connection with the superior vena cava, is positioned in this angle. The distal end of insertion piece 30 is provided with at least one hole 90. The cross-section of hole 90 should be sufficient to accommodate the flow volume in the cannula.

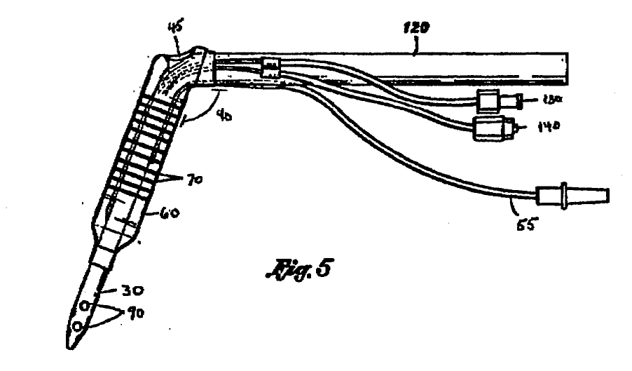

When in use, insertion piece 30 is inserted into superior vena cava, passed through right atrium, and extends into inferior vena cava. According to the invention, means for reinforcement such as metal spirals or other reinforcement means known in the art may be positioned to prevent buckling of the material from which the connecting piece 20 and the insertion piece 30 are made. Venous drainage of inferior vena cava passes through hole 90, and then through a lumen 110 in insertion piece 30, and then through a lumen 120 in connecting piece 20 (See FIGS. 3-4). Venous drainage of superior vena cava 5 passes through hole 45, and then through a lumen 120 in connecting piece 20. Connecting piece 20 is connected at its proximal end to a tube leading to a venous reservoir, not shown.

According to one embodiment of the invention, cannula 10 is provided with cooling means. These consist of an inlet for coolant 130 and an outlet for coolant 140, which are connected to a radially expandable membrane constructed of a biocompatible material. Such material may be, for example, polyurethane, silicone, latex, polyvinylchloride, polyolefin, low-density polyethylene, or polycarbonate. However, other suitable biocompatible materials known in the art may also be used. The coolant may be any suitable heat transfer fluid including, but not limited to, water, aqueous saline solution, and calcium chloride solution and perfluorocarbon fluid. One skilled in the art will realize that other fluids or gels will also be suitable for use with the present invention. The membrane has at least one unexpanded predetermined configuration and at least one expanded predetermined configuration, which may comprise a substantially cylindrical form.

When expanded, one end of the substantially cylindrical form may comprise a larger circumference than other portions. Such size differential may be used to facilitate contact with the superior and inferior vena cavae. The substantially cylindrical form may be of any length suitable for insertion. In preferred embodiments, this length is between about 6 cm and about 14 cm. The middle portion may have any diameter suitable for insertion and inflation in the desired location. However, the expanded diameter is preferably between about 15 mm and about 30 mm. The expandable membrane is in the form of an inflatable balloon 60 in one embodiment of the invention. Sections of the membrane may comprise at least two different thicknesses and may include a substantially cylindrical middle portion having at least one circumferential ring 70 formed on its inner surface. Such ring, which provides structural support, may be formed from a section of biocompatible polymer, and may preferably be formed from a thickened section of such polymer.

A passage 50 through balloon 60 is sealingly connected in fluid communication with right atrial suction tube 55. When suction is applied to tube 55, excess cardioplegic fluid used in the procedure is drained from right atrium 3 through passage 50 into tube 55, which is connected to a suction means such as a vacuum pump, not shown.

Balloon 60 may be wrapped around insertion piece 30 so that it is possible to insert it easily into superior vena cava. By applying coolant pressure in the balloon the balloon is inflated and substantially engages the interior surfaces the right atrium, superior vena cava and inferior vena cava in order to occlude and cool them. By contrast, there is little or no contact with the atrial appendages, tricuspid valve, or coronary sinus.

The expandable membrane may be produced by fabricating a substantially cylindrical mandrel having a circumference substantially equal to the circumference of the substantially cylindrical middle portion of the expandable membrane in its expanded configuration, fabricating at least one circumferential groove in the mandrel in areas corresponding to the substantially cylindrical middle portion of the expandable membrane, immersing the mandrel in a liquid dispersion of a biocompatible polymer, removing the mandrel from the dispersion, curing the biocompatible polymer on the mandrel; and, removing the solidified membrane. Alternatively, the membrane can be made by blow molding.

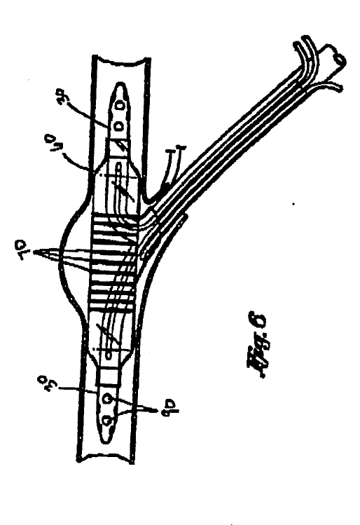

FIG. 5 is an elevational view of a cooling cannula system of the present invention, with like parts having the same numbers. In this view, it is possible to see the expandable membrane 60 and a plurality of circumferential ring 70. Such rings may be formed from a section of biocompatible polymer, and may preferably be formed from a thickened section of such polymer. Opening 45, which provides a connection with the superior vena cava, is positioned at angle 40. The distal end of insertion piece 30 is provided with at least one hole 90. As with other embodiments of the invention, the cross-section of hole 90 should be sufficient to accommodate the flow volume in the cannula.

FIG. 6 is an elevational view of a bifurcated cooling cannula system of the present invention, with like parts having the same numbers. Such embodiment may be used in the atrial region like other embodiments of the present invention. One of ordinary skill in the art will realize that this embodiment of the present invention may also be used in other regions of the body.

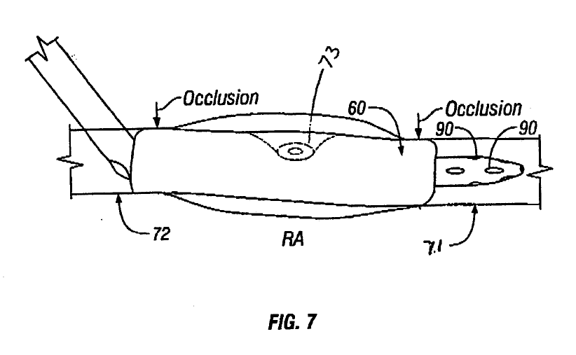

FIG. 7 is an elevational view of a cooling cannula system of the present invention, with like parts having the same numbers. Such embodiment may be inserted into the superior vena cava, thus accessing the heart without forming an incision in the right atrium. As discussed above, such atrial incisions may damage electrical conductivity pathways and produce transient conduction abnormalities. When properly placed, the cannula will block the superior vena cava and inferior vena cava inlets to the right atrium. Such isolation of the right atrium eliminates backflow at the distal anastomoses.

One of ordinary skill in the art will realize that this embodiment of the present invention may also be used in other regions of the body.

In the embodiment of the present invention shown in FIG. 7, the balloon 60 is inflated to form a seal with the surrounding inferior vena cava 71. Likewise, the balloon 60 also forms a seal with the surrounding superior vena cava 72. This occlusion of the superior vena cava and the inferior vena cava effectively isolates the right atrium and eliminates backflow at the distal anastomoses. With the backflow eliminated, addition blowers and suckers are no longer required to clear the field. Also, the surgeon does not have to reduce the amount of arterial perfusion to reduce backflow through the coronaries. This results in a flaccid, empty heart and allows for superior access to posterior graft sites which, in turn, provides a faster, cleaner surgery.

Due to the occlusion of the superior and inferior vena cava inlets, blood and cardioplegia solution can drain out of the coronary sinus and accumulate in the right atrium. The embodiment of the present invention shown in FIG. 7 also includes a drainage port 73 and line (not shown) for isolated drainage of the right atrium. This drainage port 73 and line thus allows the surgical team to isolate the additional fluid (cardioplegia solution mixed with blood) and reduce their hemodilution levels, while reducing the exposure of the patient to the potentially harmful effects of the cardioplegia solution.

As discussed above, one of the functions of the present invention is to cool the surrounding tissue during surgery. To augment heat transfer, the polymer may incorporate dispersed particles of biocompatible metal such as iron, cobalt, chromium, nickel, titanium, niobium, silver, gold, platinum, aluminum, and molybdenum, or a biocompatible metal alloy comprising at least two elements selected from the group consisting of iron, cobalt, chromium, nickel, titanium, niobium, silver, gold, platinum, aluminum, and molybdenum. Alternatively, such metal and metal alloys can be combined with the polymer in the form of layers. As used herein, the term “augment” is intended to mean that the augmented polymer transfers heat at a faster rate than untreated polymers. That is, using such metals with the polymer increases or augments the amount of heat that is transferred when compared to an un-augmented polymer.

Likewise, alternative cooling means will be well known to artisans, including at least the following; chemical by means of some manner of endothermic reaction (for example, an emergency cooling packs) and/or a mechanical, or solvent/solute based reaction set, such as by using a pre-cooled gel that requires no circulating circuit (as in a pre-frozen gel packs).

One could likewise inject chemical means, precursor means, media or related forms of an appropriate composition of matter, compound, or material (for example DMSO/RIMS050® brand as distributed by Edwards Lifesciences, Utah, USA) that has high freezing temperature. Those skilled also understand the electrical way, as in the Peltier example given, which only illustrates one of the many known techniques used recently. Such mechanisms could include a refrigerant—external compressor and evaporator and for biochemical means from iontophoresis to some way to promote lower ATP usage, or evaporative, or Conductive mechanisms to cool the air external to appropriate anatomy via Vortex tube.

Another alternative approach whereby at least one of the above cooling means could be used to cool the significant anatomy internally (a device inserted into the right atrium), or externally is within the scope of the instant teachings. All above cooling means could be combined with venous drainage cannulac or as stand-alone devices, according to the present invention.

A method of use for providing drainage of venous blood using the invention comprises opening the vasculature, draining venous blood from at least one of the right atrium, superior vena cava and inferior vena cava; and, cooling involved luminal surfaces. Drainage of venous blood and cooling of cardiac tissue can be provided from a targeted location without opening the right atrium or the chest of a patient undergoing cardiac surgery. The method involves identifying a targeted location, inserting a guidewire in a jugular vein or femoral vein of a patient, observing the placement of the guidewire fluoroscopically while passing the guidewire through the vein until the distal end of the guidewire is situated in the targeted location; threading the proximal end of the guide wire through the free end of the cannula; passing the cannula along the guidewire while observing fluoroscopically until the radiopaque area lies in the targeted location; withdrawing the guidewire; draining venous blood and cooling involved luminal surfaces. A radiopaque area (such as barium sulfate or stainless steel) may be situated near the free end of the tubular insertion section to facilitate this method.

The above-described method teaches the insertion of the present invention through a jugular or femoral vein of a patient. One of ordinary skill in the art will realize that the present invention may also be inserted through alternate peripheral vessels.

Such cooling may be used in association with known surgical techniques. Alternatively, such cooling may be used as part of a cardiac surgical procedure whereby the heart is cooled while the patient's body is maintained at a normothermic or tepid temperature. Specifically, as part of a surgical procedure, the right atrium may be cooled while the patient's body is maintained at a normothermic or tepid temperature. Such selective cooling may be referred to as a warm body/cold heart procedure. Cooling of the right atrium has the effect of also cooling the SA node and, indirectly, the AV node and the connective tissue between the SA node and the AV node. When used herein, the term ‘normothermic’ refers to a temperature that approximates the patient's normal body temperature. Such temperature is usually between 34° C. and 37° C., and typically around 36° C. Tepid refers to a temperature usually between 28° C. and 32° C. This temperature may be maintained during surgery by heating the patient's blood through means known in the art.

Also within the present invention is an article of manufacture, comprising packaging material and the novel enhanced cardiac surgical cooling system contained within the packaging material, wherein the novel enhanced cardiac surgical cooling system is effective for cardiac surgery in a patient afflicted with cardiac disease, and the packaging material includes a label that indicates that said device is effective for said cardiac surgery.

In the foregoing description, certain terms have been used for brevity, clearness and understanding, but no unnecessary limitations are to be implied therefrom beyond the requirements of the prior art. Having described preferred embodiments of the invention with reference to the accompanying drawings, it is to be understood that the invention is not limited to those precise embodiments, and that the various changes and modifications may be effected therein by one skilled in the art without departing from the scope or spirit of the invention as defined in the appended claims and their equivalents. Various additions, deletions, alterations and modifications may be made to the above-described embodiments without departing from the intended spirit and scope of the invention. It is, for example, possible to use other cooling means, such as tubing wrapped around the cannula, or other structures known in the art for efficient heat transfer, wherein insulation measures ensure that the contents of the tube are not affected. Furthermore, warming can be done instead of cooling.

1. A cardiac surgical cooling system comprising:
a generally tubular cannula adapted for placement in fluid communication with a body vessel, the cannula including an aperture effective for drainage of the superior vena cava and an aperture effective for drainage of the inferior vena cava; and
a radially expandable membrane extending circumferentially around the cannula and having at least a first configuration and a second, predetermined generally tubular expanded configuration, the cannula being configured to permit the circulation of coolant within the biocompatible membrane,
wherein the portion of the predetermined configuration that lies within a right atrium fills a volume that is smaller than the volume of the right atrium.

2. The cardiac surgical cooling system according to claim 1, wherein the cannula in its expanded configuration comprises a first portion that occludes the superior vena cava and a second portion that occludes the inferior vena cava.

3. The cardiac surgical cooling system according to claim 1, wherein the biocompatible membrane has a first portion, a second portion and a central portion lying between the first and second portions, and the central portion has a circumference that is the same or smaller than the circumference of either the first or the second portion.

4. The cardiac surgical cooling system according to claim 1, wherein said biocompatible membrane is a polymer.

5. The cardiac surgical cooling system according to claim 4, wherein said polymer is selected from the group consisting of polyurethane, silicone, latex, polyvinylchioride, polyolefin, low density polyethylene, polycarbonate, polymers that have a metal layer formed on one surface, polymers that have a metal layer formed between an interior surface and an exterior surface, and polymers having metal dispersed throughout them.

6. The cardiac surgical cooling system according to claim 1, wherein the biocompatible membrane includes a material selected from the group consisting of a metal and a metal alloy.

7. The cardiac surgical cooling system according to claim 1-, wherein the biocompatible membrane comprises a material selected from the group consisting of steel, iron, cobalt, chromium, nickel, titanium, niobium, silver, gold, platinum, aluminum and molybdenum.

8. The cardiac surgical cooling system according to claim 1, wherein said expandable membrane includes a substantially cylindrical middle portion having at least one circumferential ring formed on its inner surface by thickened sections of the membrane.

9. The cardiac surgical cooling system according to claim 1, wherein said expandable membrane has an inlet connection for supplying said heat transfer fluid to said expandable membrane, and an outlet connection for draining said heat transfer fluid from said expandable membrane.

10. A method for making the expandable membrane as defined in claim 8, said method comprising the steps of:
fabricating a substantially cylindrical mandrel having a circumference substantially equal to said circumference of said substantially cylindrical middle portion of said expandable membrane in said expanded configuration;
fabricating at least one circumferential groove in said mandrel in areas corresponding to said substantially cylindrical middle portion of said expandable membrane;
immersing said mandrel in a liquid dispersion of said biocompatible polymer;
removing said mandrel from said liquid dispersion of said biocompatible polymer;
curing said biocompatible polymer on said mandrel; and
removing said solidified membrane.

11. A method for making the expandable membrane as defined in claim 8, said method comprising the steps of:
fabricating a mold adapted for blow molding said expandable membrane in said at least one expanded predetermined configuration; and,
blow molding said expandable membrane.

12. The cardiac surgical cooling system according to claim 1, wherein the cannula further includes a drainage port configured to drain the right atrium.

13. The cardiac surgical cooling system according to claim 1, wherein the drainage port is located between the aperture for drainage of the superior vena cava and the aperture for drainage of the inferior vena cava.

14. The cardiac surgical cooling system according to claim 1, wherein the system defines an axis about which a heart may be rotated.

15. The cardiac surgical cooling system according to claim 1, wherein the cooling system further comprises a radiopaque area situated adjacent to a distal end of said cannula.

16. A cannula configured to cool tissue and drain venous blood from at least one of the inferior vena cave, the superior vena cava and the right atrium, the cannula comprising:
a connecting piece having a proximal end, a distal end and an internal lumen through which venous blood may flow, wherein the connecting piece is configured for connection to a suction device;
a generally tubular insertion piece coupled to the connecting piece and configured for insertion into a body vessel, the insertion piece having a proximal end, a distal end and a lumen, the insertion piece further defining an opening adjacent to the distal end through which venous blood may enter the lumen of the insertion piece, wherein the proximal end of the insertion piece is configured to be in fluid connection with the connecting piece at a junction, the junction defining an opening through which venous blood may flow into the lumen of the connecting piece; and
a generally tubular, radially expandable portion circumferentially disposed about the insertion piece, wherein the radially expandable portion has a first portion, a second portion and a central portion lying between the first and second portions, and the central portion has a circumference that is the same or smaller than the circumference of the second portion.

17. A method of performing cardiac surgery comprising the steps of:
inserting a cannula into a patient's right atrium, said cannula configured to cool body tissue;
draining venous blood from at least one of the right atrium, superior vena cava, and inferior vena cava; and
cooling at least a portion of the right atrium while maintaining the majority of the patient's body at a normothermic temperature.

18. The method of claim 17, wherein the cannula has at least one aperture effective for direct drainage of a body vessel, and further wherein the cannula is in fluid communication with a suction means for applying reverse pressure.

19. The method of claim 17, wherein the cannula further includes a generally tubular radially expandable biocompatible membrane extending circumferentially around the cannula and having at least a first and a second configuration.

20. The method of claim 17, wherein the cannula is configured to permit the circulation of coolant within the biocompatible membrane.

21. The method of claim 17, further including the step of heating the drained venous blood and reintroducing the drained venous blood into the patient.

22. The method of claim 17, wherein the cooling step comprises cooling a portion of the right atrium to a temperature below 20° C.

23. The method of claim 17, wherein the cooling step comprises cooling a portion of the right atrium to a temperature between 10° C. and 15° C.

24. The method of claim 17, wherein the majority of the patient's body is maintained at a temperature between 34° C. and 37° C.
